(19) United States
(12) Patent Application Publication
(10) Pub. No.: US 2020/0268961 A1
(43) Pub. Date: Aug. 27, 2020

(54) HEMODYNAMIC SWIRLING OF EXTRACORPOREAL BLOOD

(71) Applicant: Asher Milgrom, Irvine, CA (US)

(72) Inventor: Asher Milgrom, Irvine, CA (US)

(21) Appl. No.: 16/428,303

(22) Filed: May 31, 2019

Related U.S. Application Data
(60) Provisional application No. 62/808,622, filed on Feb. 21, 2019.

(51) Int. Cl. A61M 1/36; A61M 1/02

(52) U.S. Cl. CPC A61M 1/3672; A61M 1/025; A61M 1/367

(57) ABSTRACT

A device for maintaining blood substantially free of biochemical coagulation and/or particulate precipitation, during extracorporeal treatment of blood is disclosed. Also disclosed are methods of using the device in an extracorporeal treatment of blood and kits comprising apparatus for performing such a method.

### RELATED APPLICATIONS

The present application claims priority to pending U.S. Provisional Patent Application 62/808,622, filed on Feb. 21, 2019, the entire contents of which are hereby incorporated by reference.

### BACKGROUND

There are various medical treatments that require blood to be removed from the body, stored temporarily in an appropriate container or bag, treated, and then returned to the body (re-infused). During the time that the blood is held in the container, if static, the blood tends to clot via inherent biochemical coagulation pathways, and particulate matter tends to separate from solution and collect gravitationally at the bottom of the container which can create a plug/clog in the outlet from the container or in a downstream filter. Such coagulation and/or clogging can disrupt the proper performance of the medical treatment or result in complications to the patient.

For example, two such treatment devices that infuse extracorporeal blood with ozone are the 10-pass device from Herrmann Apparatebau GmbH and the device from Zotzmann & Stahl GmbH. Operators of both devices often encounter this clogging problem, that disrupts the procedure, requiring changing of parts the apparatus. To combat this tendency to clog, the manufacturers of both devices instruct the users of the device (doctors) to utilize anticoagulant substances such as heparin.

Furthermore, attendant IV nurses or doctors have learned that manually and continuously swirling the containers can marginally help keep the apparatus from clotting/clogging.

Plasmapheresis and treatment of blood with UV-B radiation are also known extracorporeal treatments of blood, among others.

Presently disclosed are apparatus and associated methods for preventing coagulation of blood or precipitation of insoluble material during an extracorporeal treatment, thus avoiding problems relating to such coagulation and precipitation.

### SUMMARY

Accordingly, one aspect of the present disclosure is a device, herein a “Hemodynamic Swirling Apparatus” or “HSA”, for maintaining blood substantially free of biochemical coagulation and/or particulate precipitation, during extracorporeal treatment of the blood. The device can be one comprising:
i. a cradle for holding a blood container securely during an orbital rotary motion about a vertical axis;
ii. a motor connected to the cradle for generating a circular orbital rotary motion the cradle about a vertical axis.

Another aspect of the present disclosures is a method for maintaining blood in a state of laminar hemodynamic flow over a blood-contacting surface and substantially free of coagulating blood and/or particulate precipitation. Such a method can be one comprising:
a. introducing a volume of the blood into a blood container; and
b. moving the container in a rotary orbital motion to generate a sinusoidal wave (or an approximately sinusoidal wave) in the moving blood thus establishing a laminar flow of the blood in the blood container and over the blood-contacting surface.

Apparatus such the device or other apparatus used in a method disclosed herein can conveniently be held by a shelf article that is a further aspect of the disclosure. Such a shelf article can be one comprising:
i. a shelf;
ii. a foreplate comprising a first half-collar for surrounding a pole, and a supporting member for supporting a shelf member placed thereon; and
iii. backplate comprising a second half-collar for surrounding a pole;
   wherein the fore-plate and backplate are configured to be attached to one another such that the first half-collar and the second half-collar form a channel to substantially surround the pole.

Kits comprising apparatus and other materials for performing a procedure of an extracorporeal blood treatment are also disclosed. Such a kit can be one comprising a cradle for holding a blood container securely during an orbital rotary motion about a vertical axis, and a motor configured to be connected to the cradle for generating a circular orbital rotary motion the cradle about a vertical axis. Such a kit can further comprise one or more adaptors for connecting the motor to the cradle.

Such a kit can be one comprising the device of claim 1, and a shelf article comprising:
i. a shelf;
ii. a foreplate comprising a first half-collar for surrounding a pole, and a supporting member for supporting a shelf member placed thereon; and
iii. backplate comprising a second half-collar for surrounding a pole;
   wherein the fore-plate and backplate are configured to be attached to one another such that the first half-collar and the second half-collar form a channel to substantially surrounds the pole.

### BRIEF DESCRIPTION OF THE DRAWINGS

While the specification concludes with claims, which particularly point out and distinctly claim the subject matter described herein, it is believed the subject matter can be better understood from the following description of certain examples taken in conjunction with the accompanying drawings, in which like reference numerals identify the same elements and in which:

FIGS. 1A and 1B show an embodiment of a cradle and adaptor as disclosed herein.

FIG. 2A shows one embodiment of a vortex motor as described herein. FIG. 2B shows a second embodiment of a vortex motor as described herein.

FIGS. 3A to 3F show an embodiment of a shelf as described herein.

FIG. 4A shows an assembled Hemodynamic Swirling Apparatus as described herein placed on a shelf as described herein. FIG. 4B illustrates the Hemodynamic Swirling Apparatus holding a blood container as in operation, showing the approximately sinusoidal shape formed by the blood as it moves within the container.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient,” “host,” “user,” and “subject” refer to any human or animal subject and are not intended to limit the systems or methods to human use, although use of the subject invention in a human patient represents a preferred embodiment.

The mammalian circulatory system works in a regime of laminar flow (high Reynold's number), not turbulent flow (low Reynold's number). Generally, in larger blood vessels blood moves in “hemodynamic laminar flow”, in which there are multiple concentric layers of blood flowing at different velocities; fastest in the middle, slowest at the blood vessel walls. If the blood vessel becomes damaged, or plaque builds up on the walls, the flow becomes progressively more turbulent. Subsequently, pressure gradients, eddies etc. are created which are a problem; contributing to emboli, accelerated buildup of additional plaque, and a potential environment for festering infections, as in infective endocarditis etc.

Laminar flow contributes to the blood's ability to keep its constituents in solution, whereas turbulent flow results in some blood substances, for example, lipids, cholesterol, chylomicrons, certain protein carrying molecules that are very large, albumin, and the like, precipitating out of solution. The more the blood is subjected to turbulence, the greater the probability of coagulation and/or precipitation of a blood constituent.

Laminar flow also contributes to maintaining blood in an uncoagulated state. It is well known that stagnant blood begins to clot; laminar flow keeps the blood in motion and inhibits coagulation. To the degree that the coagulation activity of the blood in a container is inhibited as well as the coagulation activity of the blood is inhibited by a clinically relevant dose of anticoagulant (e.g., heparin), the flow in the container is maintaining the blood in an uncoagulated state.

The coagulation state of a blood sample can be measured by methods known in the art, for example by thrombin time. Use of an HSA as disclosed herein in a method of treating blood extracorporeally typically provides blood flowing out of the outlet of a blood container used with a HSA that has a thrombin time substantially the same as the blood flowing into the blood container.

The Hemodynamic Swirling Apparatus (or “HSA”) disclosed herein simulates hemodynamic laminar flow within a container, which keeps blood from coagulating, and blood constituents in solution. An orbital motion is applied by a motor of the Hemodynamic Swirling Apparatus and the orbital motion keeps the blood and its constituents flowing in a single direction throughout a blood container in which blood is exposed to a treating substance (or in which a substance is removed from the blood), the blood constituents staying in solution and not forming a plug/clog at the bottom of the container or in a downstream filter or component. By the circular rotary motion, the blood in the blood container maintains a laminar flow throughout the container and over the surface sufficiently that blood in the blood container does not coagulate to the degree that it causes clotting or clogging of the container outlet, or its filters and tubing, necessitating the replacement of clogged elements with fresh ones.

The HSA causes a centrifugal laminar flow that produces a smooth sinusoidal wave (or an approximately sinusoidal wave) in the blood flowing around the blood container. This sinusoidal wave can be visualized as a wave shape at the top edge of the blood moving in a transparent blood container. When a laminar flow in the blood container is established, a sinusoidal wave is seen at the top edge of the blood moving in the blood container that will keep its shape as it moves around the container. The appearance of this wave can be used to “tune” the orbital motion of the Hemodynamic Swirling Apparatus to the particular blood container being used, for example by small variation in the orbital rpm. It might be necessary additionally or alternatively to adjust the diameter of the orbit when the HSA is used with some configurations of the blood container.

A HSA keeps extracorporeal blood in a state of hemodynamic laminar flow, eliminating clinical problems caused by biochemical coagulation and/or particulate clogging. For example, in known ozone treatments, a significant problem of clogging of a filter at the outlet of the ozone-exposing blood container requires frequent changing of the blood container. This problem is typically addressed by use of substantial amounts of heparin and even then is not always entirely successful, frequently necessitating replacing the clogged elements with fresh ones. Furthermore, the use of heparin or other anticoagulants raises health risks of its own in patients for whom anticoagulants are contraindicated.

Two marketers of apparatus for use in extracorporeal treatment of blood are Herrmann Apparatebau GmbH and Zotzmann & Stahl GmbH. Both instruct use of anticoagulants such as heparin during use of their apparatus to prevent clogging of elements of the apparatus in use.

A Hemodynamic Swirling Apparatus comprises an orbital vortex motor that “swirls” a container in a circular orbital motion. The orbital diameter can be from 6 mm to 22 mm depending on the shape and size of the container. The orbital diameter is typically from 6 to 22 mm, but can be from 12-19 mm, or from 16-18 mm. The orbital speed can be from 140 rpm to 165 rpm depending on the size and shape of the container, and is usually from 145 to 165, from 155 to 165 rpm, or from 160 to 165 rpm.

Control of the orbital speed is important to the quality of the laminar flow in the moving blood. A motor having sufficient torque to maintain the desired rpm of the orbital platform under the changing load of blood volume as the blood container is filled (or emptied) is required. The motor is preferably one that accurately and digitally maintains the set rpm of the orbital motion is preferably under control of a microprocessor that monitors the orbital motion and digitally adjusts the motor output accordingly. For purposes of this application, design and implementation of such microprocessors are considered known in the art.

When the motor is turned on, the speed of the motor will preferably ramp up gently, preferably the ramp up time from 0 rpm to 160 rpm should be between 3-7 seconds. Such gentle ramp up of motor speed minimizes any unexpected ejection of the blood container from the cradle by a sudden acceleration, and also prevents shock or turbulence to any blood present in the blood container at start-up, which might induce clotting.

The orbital vortex motor can be configured with an attachment assembly that firmly holds the cradle (described below) and allows for cradle replacement if needed.

The orbital vortex motor can be configured with leveling apparatus and “feet” that are height adjustable to be able to adjust the motor to a level aspect such that the axis of the orbital motion of the motor is vertical. Such a leveling apparatus can be one that allows for reading level about two axes.

A Hemodynamic Swirling Apparatus further comprises a cradle that holds a blood container. The cradle can be any configuration that tightly holds and firmly fits the blood container and does not allow any “rattle” of the blood container in the cradle in operation of the Hemodynamic Swirling Apparatus. An elastomeric element can be fitted to a portion of the cradle holding the blood container to assist in providing a snug fit of the blood container and prevent rattling. Preferably, the cradle should provide easy insertion and removal of the container from the cradle with one hand.

The cradle includes at least one ring structure having a central cavity for encircling the container. The ring structure can form an incomplete ring, providing a gap in the ring through which tubing and other items trailing from the blood container can easily be passed in and out of the ring. The gap in the ring might be closable by an appropriately configured ring-closing member, such a section of the circumference attached to one end of the gap in a pivotable manner. The at least one ring is fixed to a bottom member that is configured for attachment to the rotary element of the orbital vortex motor and for attachment to a support structure for holding the incomplete ring members.

The cradle is attached to the motor either directly, or preferably through an adaptor piece. The adaptor is configured to receive the bottom member of the cradle and to engage the motor element (“head”) that moves the cradle in a circular orbit. In some embodiments, the adaptor can be secured to the motor head by a friction fit, by a screw, or any other sort of attachment that provides that the adaptor can be removed from the motor head. Additionally or alternatively, the cradle can be removably attached to the adaptor, which in such embodiments might be either permanently or removably attached to the motor head. Such removable attachments permit changing of cradles, for example, for receiving different configurations of blood containers.

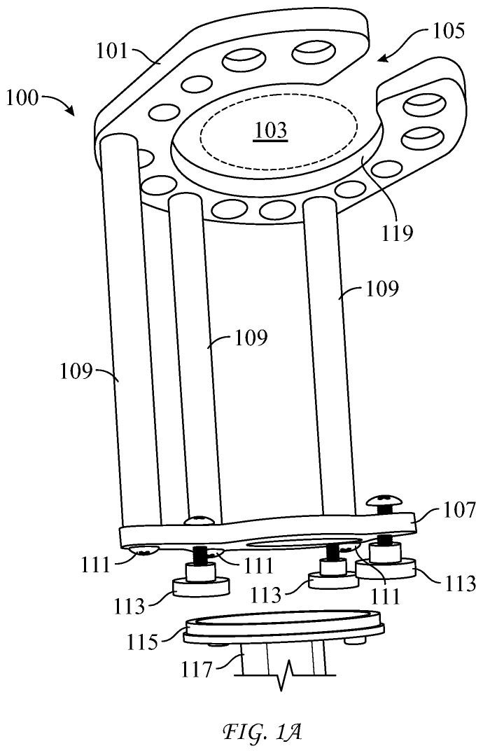

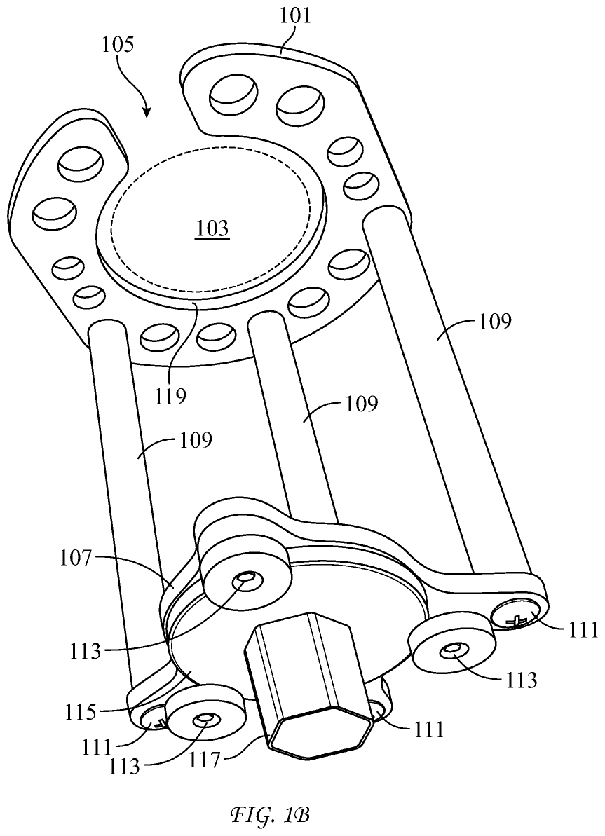

One embodiment of a cradle is illustrated in FIG. 1A. In such an embodiment a cradle 100 comprises a top member that is an incomplete ring structure 101 having a central aperture 103 for encircling the container and providing a gap 105 in the top member of the cradle. A second ring structure is used as the bottom member 107 of the cradle. The top and bottom members are separated one from the other and supported over the bottom member by one or more (3 in FIG. 1A) joining members 109 that in this embodiment are elongated tubes attached to each of the top and bottom members. In the illustrated embodiment, the joining is by screws 111. Holes are disposed equidistantly along the ring of the bottom member through which hand-tightenable screws 113, that are used to attach the cradle to an adaptor 115 for attachment to the orbital vortex motor, can be disposed. The corresponding adaptor 115 for affixing the cradle to the motor includes correspondingly-spaced holes for receiving the screws 111 (not shown) and comprises a central shaft 117 that can be fit to a correspondingly shaped member of a member on the vortex motor that provides an orbital motion.

FIG. 1B shows a bottom view of the cradle assembled onto the adaptor. The hexagonal void in the shaft portion of the adaptor is apparent.

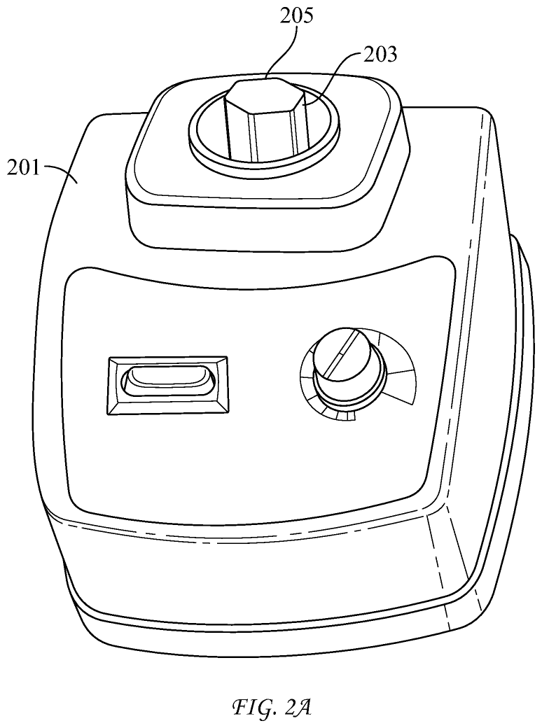

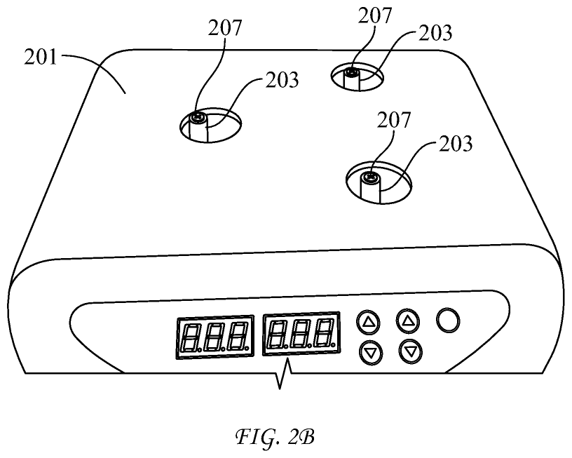

FIG. 2A illustrates a vortex motor. The motor 201 includes a member for moving an attached part in a circular orbital motion 203. In the illustrated embodiment this member has a hexagonally-shaped outer surface that fits tightly to the hexagonal void in the adaptor 115. The member 203 also has a centrally-tapped screw thread 205 for receiving a screw to fasten the adaptor to the member 203 securely.

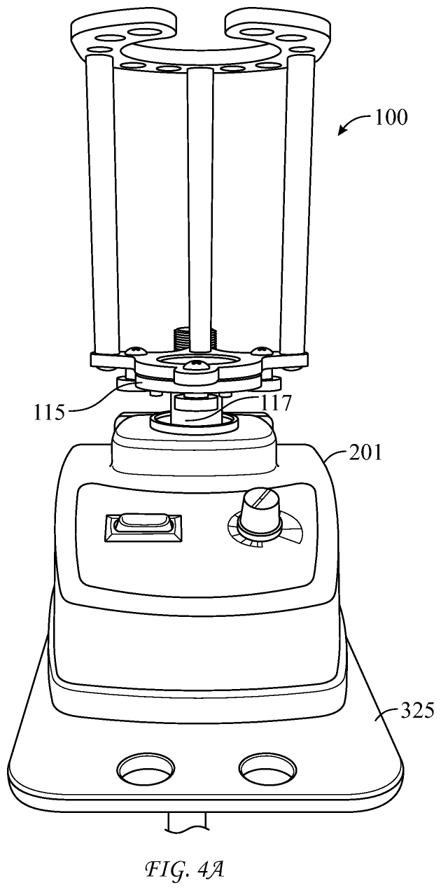

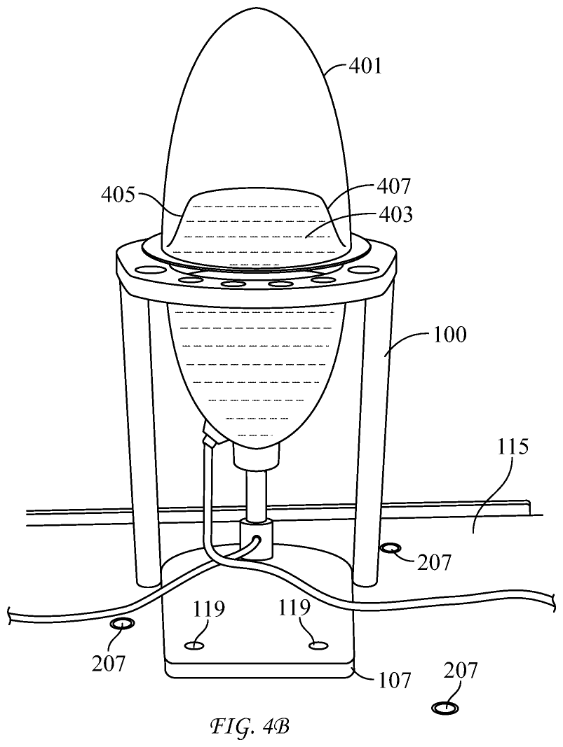

FIG. 2B shows another embodiment of a vortex motor. In this embodiment, the vortex motor 201 is a more powerful one than the embodiment in FIGS. 2 and 4A. In this embodiment, the vortex motor 201 includes a plurality of head members 203 that move together to provide a circular orbital motion. Each of the members 203 has a centrally tapped thread by which an adaptor having the form of a plate can be securely and removably attached by a screw 207.

The various members of the cradle can be fabricated from any suitably rigid material. The cradle as a whole is preferably light in weight. So, if made from a metal, preferably a member is made from aluminum or titanium. If made from a plastic, polycarbonate and poly(meth)acrylates can be used.

Members that contact the container can include in their portions contacting the container an elastomeric material, to provide a surface that can effect a tight friction fit, of the container. For instance, again referring to FIGS. 1A, 1B, the central cavity of the top member might be faced with a silicone gasket or tape wrapped over the inner edge 119. A member formed of metal can be dipped into a melted elastomer material.

A Hemodynamic Swirling Apparatus is used together with or can further comprise a rigid blood container that has a circular cross section in the plane of the orbital axis. Thus, the blood container can be one having a spherical, an oblate spheroid, circular cylindrical or circular conical shape and comprising;
a. at least one fitting configured to receive a volume of blood into the container, preferably via a transport line;
b. at least one fitting configured to deliver a volume of blood from the container into a transport line;
c. a third fitting configured for introducing a substance to treat the blood.
   In some embodiments, the fittings a, and b, can be the same or can connect to the blood container through the same opening. That is, the blood container can be filled or emptied via the same fitting or through the same opening.

The blood container preferably has a more or less constant diameter of cross section. Taper of the cross section at the top and/or bottom of the blood container is acceptable, but is preferably small in extent and not steep in gradient.

The shape of the blood container should be one that minimizes shear stress from the top to the bottom of the blood container in the blood moving in the blood container during operation.

The diameter of the blood container should such that there is not too much variation in the rate of flow of the blood around the blood container in use. A container having too large a diameter will result in too much variation in flow velocity radially from the wall of the container toward the center. Extreme variations in velocity can create turbulence, which can induce coagulation. Too small a diameter will result information of vortices in the flowing blood (like a “whirlpool”) that is likely to cause turbulence and induce coagulation. A container having a diameter of 4 to 6 inches at its widest point is preferred.

A blood container is preferably made from a blood-compatible material, such as a glass, for example a silicon oxycarbide glass or glass coated with sulfobutaine or carboxybetaine polymers. Containers provided by Zotzmann & Stahl GmbH are made from a kind of glass. Plastic containers for use in some extracorporeal blood treatments are known. For example a blood container suitable for use with a Hemodynamic Swirling Apparatus as disclosed herein is made from polycarbonate and available from Hermann Apparatebau GmbH. Polyvinyl chloride, and silicone-based plastics can also be used to fabricate a blood container for use with a Hemodynamic Swirling Apparatus.

A blood container for use with the Hemodynamic Swirling Apparatus preferably will not have any seams in the blood contacting surface that extend across the orbit of blood moving around the blood container, as such seams tend to create turbulence.

Preferably a blood container for use with the HSA will be inert to a substance or radiation that is used to treat the blood in the container. For instance, the blood container might be made of a material that is inert to ozone.

In use, a Hemodynamic Swirling Apparatus can be placed on any appropriate horizontal surface adjacent to any other apparatus that might be used in the extracorporeal blood treatment. For example, next to an ozonation device in a procedure that contacts blood with ozone.

The Hemodynamic Swirling Apparatus can be placed on any convenient horizontal, level surface. The HSA can be conveniently placed on a shelf that is part of a cart or on a shelf that is attached to a IVpole holding other items used in a procedure for administering or removing a substance from intravenous blood (described herein).

Thus, a Hemodynamic Swirling Apparatus can be provided in a kit form comprising a Hemodynamic Swirling Apparatus and a shelf for holding the HSA during use, the HSA comprising:
a cradle for holding the container securely during an orbital rotary motion about a vertical axis; and
a motor for generating an orbital rotary motion having an axis parallel to the vertical or long axis of the container.

A kit can further comprise a shelf configured to attach to an IV pole to hold the HSA, and/or a shelf designed to hold the HSA that is an integral part of a cart designed to hold the other components needed for said procedure.

A HSA can be provided in separate parts. A kit can include a motor and a cradle, and optionally further include one or more adaptors, as separate pieces. Such a kit can further include instructions for use of the kit items in a procedure that includes a step of extracorporeal treatment of blood with a substance or a form of radiation. A further addition or alternative to the parts above can be a blood container as described above.

In such a kit, the shelf can be one designed to attach to an IV pole on which all manner of items can be placed, including the HSA vortex motor. In some embodiments, the shelf is provided alone. The shelf can be one comprising a fore-plate and a backplate. The fore-plate comprises a first member that forms a portion of a collar for surrounding a pole that can be tightened against the pole and a second member extending therefrom, generally perpendicularly to the first member that supports the shelf. The fore-plate can be formed from two pieces, each of which is a mirror image of the other and joined together at their supporting members, e.g. by rivets or screws or welding. The fore-plate further comprises a third “shelf” member, which can be the shelf itself or to which the shelf can be attached. The third member extends generally perpendicularly from the top of the support member of the fore-plate.

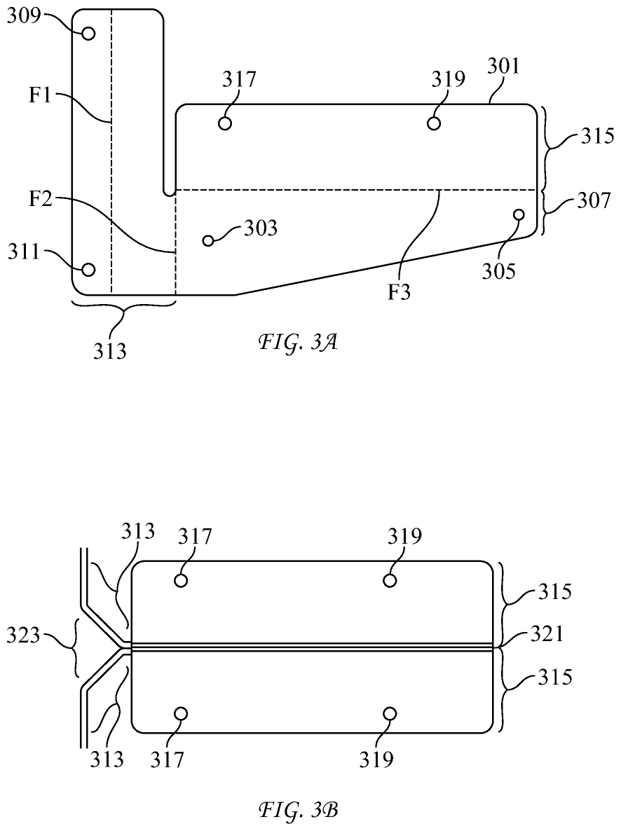

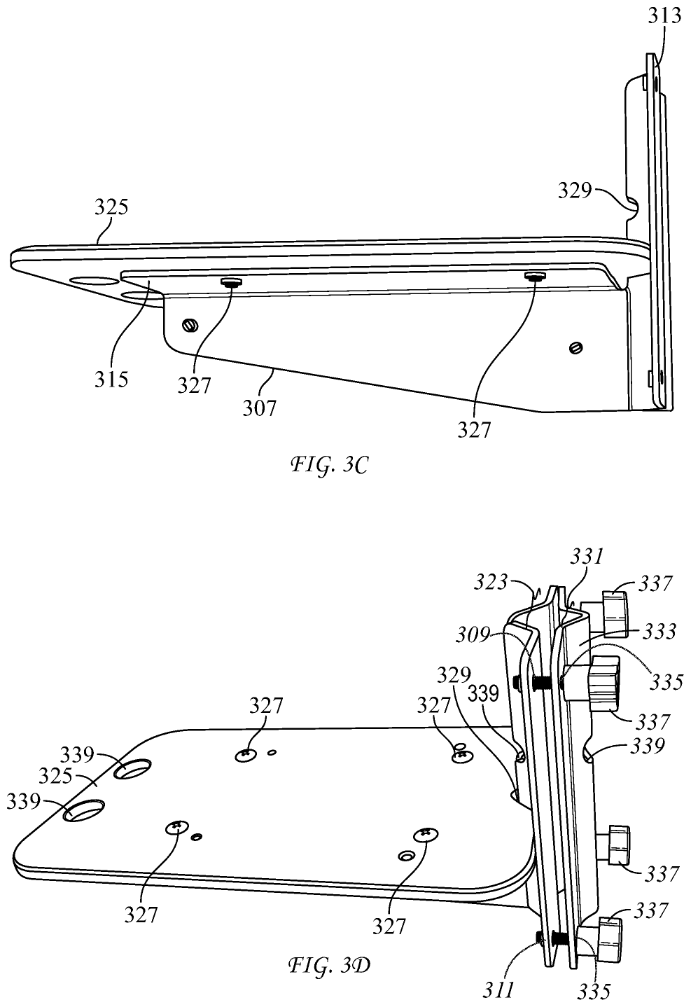

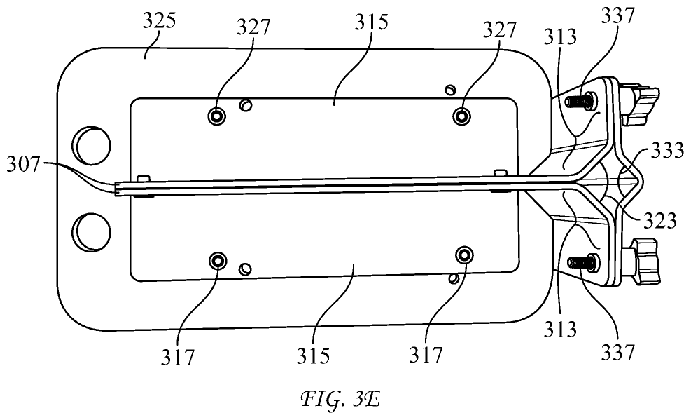

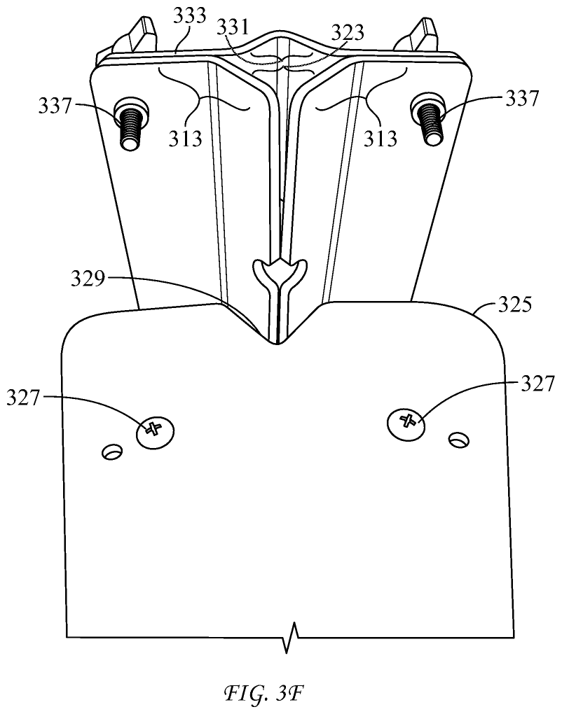

In one embodiment, the fore-plate is fabricated from a metal in a two flat pieces such as illustrated in FIGS. 3A and 3B. In FIG. 3A, a flat plate that is cut to shape 301 is perforated by holes 303 and 305 through a supporting member portion 307 for joining two such plates, after bending to the proper form, by rivets or screws or a bolt. The plate is also perforated by holes 309 and 311 in a collar-forming portion 313 for joining to a back plate to affix the shelf to a pole.

One half of a fore-plate is formed by bending the plate along the fold lines F1 and F2 to oblique angles to form the collar portion, and by folding the plate along the fold line F3 to be perpendicular to the support member to form a shelf member from a shelf member-forming portion 315. Screw holes 317 and 319 penetrate the shelf member for attaching a shelf. In some embodiments, the shelf members themselves are used as a shelf.

FIG. 3B shows a top view of an assembled fore-plate. The completed fore-plate is made by joining a half of a fore-plate to a second half of a fore-plate bent to form a mirror image of the first half fore-plate. Then the two halves are joined by fixing one to the other along the support member. In FIG. 3B, the edge of the joined surfaces of the support member form the line 321. The collar is formed by the collar-forming portions 313. The shelf support is formed from the shelf-forming members 315. The screw holes 317 and 319 are shown. The half-collar 323 is formed by the two collar-forming portions of the plates 313.

The shelf itself can be formed either as an integral part of the fore-plate support member, or can be a separate piece that is attached to the support member at the shelf member. In instances where the shelf is a separate piece, it can be made from any suitable material for supporting items, and is preferably light in weight. Generally the shelf can be made from any rigid plastic or metal so as to be durable and easy to clean. The shelf might be covered on top by a slightly resilient and/or slightly textured material to provide for a surface that is not slippery. The shelf can include attachment points, such as screw holes or loops or slots, for securing items placed on the shelf.

FIG. 3C shows a side view of a shelf 325 attached to a fore-plate. The shelf is fixed to the shelf-member 315 of the fore-plate by screws 327. The half-collar portion 313 of the fore-plate for fixing the shelf to a pole is shown.

FIG. 3D shows an example of a shelf 325 fixed to a support by screws 327. The shelf includes a notch 329 configured to fit over a matching convex shape formed in the half-collar of the fore-plate. The half-collar of the fore-plate is in the illustrated embodiment mirrored in the concave half-collar 331 shaped into a backplate 333.

FIG. 3E shows a bottom view of an assembled shelf and support. In FIG. 3E, the shelf 325 is fixed to the shelf members of the fore-plate 315 by screws 327. The supporting members of the fore-plate 307 are shown fixed to one another by rivets. The manner in which the half-collar forming members of the fore-plate 313 form the half-collar 323 and their combination with the half-collar 333 of the backplate to surround a pole is apparent. The hand tightening screws 337 used to fix the backplate to the fore-plate around a pole are shown.

FIG. 3F shows a top view of the assembled shelf and support. In FIG. 3F, The shelf 325 is fixed to the shelf members of the fore-plate 315 by screws 327.

The assembled fore-plate (before or after attaching the shelf) can be attached to a pole, such as an IV pole, using a backplate as illustrated in FIG. 3C. The backplate can be made of any suitably rigid material, in the illustrated instance, a metal, and is formed into a second half-collar 333 and is perforated by holes 335. In use, holes 335 are threaded, together with the holes 309 and 311 in the fore-plate, by hand-tightenable screws 337 to form a collar around a pole.

The shelf, the shelf member of the fore-plate and/or the half-collar portions of the fore-plate and backplate can be pierced by holes 339 that allow the passage of cables or tubes or the like, or pins or screws or other devices for holding a shelf in place on a pole, through them. The holes can also hold syringes or other items conveniently to the procedure taking place.

A kit can be one including (the parts of) a Hemodynamic Swirling Apparatus together with a shelf as described above. In some embodiments, kits can also include a blood container as described above.

FIG. 4A shows an embodiment including a shelf assembled on an IV pole and supporting a vortex motor having a cradle attached thereto with an adaptor, all as described above. Cradle 100 is attached to the adaptor 115, which is in turn secured to the head portion of the motor 201 by screws 207. The assembled HSA sits upon shelf 325.

FIG. 4B shows a second embodiment of an assembled HSA, this particular one sitting on a countertop. In FIG. 4B, a plate-form adaptor 115 is fixed to the head portions of the motor (not shown). A plate-form adaptor can be made of a variety of materials, for example a metal such as stainless steel or aluminium, or a plastic such as polycarbonate, and includes holes or other means positioned appropriately to receive an attachment device of a cradle 100 via the bottom plate 107 of the cradle. In the illustrated embodiment, the plate can receive one cradle, but in some embodiments, the plate-form adaptor can be configured to receive more than one cradle. In the illustrated embodiment, the cradle is attached to the plate-form adaptor by screws run through through-holes 119 in the bottom member of the cradle and into corresponding holes in the adaptor. In some embodiments, the screws can be secured by threads in the through-holes, and/or by a nut.

FIG. 4B also shows a blood container 401 held by the cradle 100. In motion, blood 403 in the container moves around the container in a wave having an approximately sinusoidal shape with leading edge 405 and trailing edge 407.

One might consider that the rotary motion of the Hemodynamic Swirling Apparatus, especially when it is filled with a substantial volume of blood, might induce a translational motion of the entire apparatus upon which the HSA is resting, especially in an instance when the HSA rests on a shelf attached to an IV pole, which typically has a wheeled stand. Such translation motion of the apparatus during use is preferably avoided, and so for instance, an IV pole to which a shelf supporting a HSA is attached will preferably be one having wheels that can be locked to prevent rolling, or one that lacks any wheels and sits firmly on a floor, or is attached to wheeled cart preferably one with locking wheels, or attached to a stationary object.

A Hemodynamic Swirling Apparatus as described herein is typically used in an extracorporeal blood ozonating treatment as follows:

1. An IV puncture and vein access is established, and the tubing from the vein is connected to an inlet to the blood container. Then the blood container is placed in the cradle portion of the HSA.

2. The HSA is activated. (Once activated the HSA typically does not have to be touched again until the treatment is completed.)

3. The ozonating device applies suction to draw the blood from the patient into the container.

4. Once the appropriate amount of blood is sucked into the container, the suction is stopped and the ozonation device introduces a precise oxygen/ozone mixture into the container, thereby ozonating the blood.

5. When the ozonation phase has been completed, the blood is pumped back into the patient.

6. Steps 3-5 constitute a single “pass” and are repeated up to 10 times.

A Hemodynamic Swirling Apparatus might be used in any extracorporeal blood treatment protocol to maintain the blood in hemodynamic laminar flow to prevent coagulation or precipitation of insoluble substances during the treatment protocol.

Additionally, the HSA might also be used as a means of mixing a particular compound into extracorporeal blood as an integral part of a prescribed therapy.

### EMBODIMENTS

A Hemodynamic Swirling Apparatus and methods of use of a HSA, and kits for performing such methods including a HSA as described herein can be embodied as set forth below.

### Embodiment 1

A device for maintaining blood substantially free of biochemical coagulation and/or particulate precipitation, during extracorporeal treatment of the blood comprising:
i. a cradle for holding a blood container securely during an orbital rotary motion about a vertical axis;
ii. a motor connected to the cradle for generating a circular orbital rotary motion of the cradle about a vertical axis.

### Embodiment 2

The device of embodiment 1, that further comprises an adaptor attaching the cradle to the motor.

### Embodiment 3

The device of embodiment 1 or 2, wherein the motor is configured to operate with an orbital diameter of from approximately 6 mm to approximately 22 mm and a rotational rate of from approximately 140 rpm to approximately 165 rpm.

### Embodiment 4

The device of any one of embodiments 1-3, in which the cradle comprises

an upper ring, including a circumferential body and an aperture, the aperture being configured to hold a blood container, the upper ring further optionally including an opening in the circumferential part of the ring;

a bottom member spaced apart from the upper ring and including or having attached thereto at least one member for operably connecting the bottom member to the motor or to an adaptor;

at least one member joining the upper ring and bottom member.

### Embodiment 5

A method for maintaining blood in a state of laminar hemodynamic flow over a blood-contacting surface and substantially free of coagulating blood and/or particulate precipitation comprising:
a. introducing a volume of the blood into a blood container; and
b. moving the container in a rotary orbital motion to generate a sinusoidal wave in the moving blood thus establishing a laminar flow of the blood in the blood container and over the blood-contacting surface.

### Embodiment 6

The method of embodiment 5, wherein the container is a rigid container having an oblate spheroid, circular cylindrical or circular conical shape.

### Embodiment 7

The method of claim 5, in which the container has a circular cross section in a plane perpendicular to the vertical axis and through the widest portion of the container has a diameter from 4 inches to 6 inches, and the diameter of the rotary orbital motion is from 6 mm to 22 mm.

### Embodiment 8

The method of any one of embodiments 5-7, in which the orbital motion has a rate of approximately 140 to approximately 165 rpm.

### Embodiment 9

The method of any one of embodiments 5-8, in which the container is one wherein a blood-contacting surface of the container comprises a glass, a polycarbonate plastic, a polyvinyl chloride plastic or a silicone-based plastic.

### Embodiment 10

The method of any one of embodiments 5-9, in which the blood is contacted with ozone gas.

### Embodiment 11

In a method for extracorporeal treatment of blood, the improvement comprising maintaining the blood in a state of unidirectional hemodynamic laminar flow substantially free of biochemical coagulation activity and/or particulate precipitation by applying an orbital rotary motion to a volume of the blood to generate a sinusoidal wave in the moving blood thus establishing a laminar flow of the blood throughout the container and over a blood-contacting surface.

### Embodiment 12

The method of embodiment 11, in which the orbital diameter is from 6 to 22 mm.

### Embodiment 13

The method of embodiment 11 or 12, in which the orbital rotary motion is a circular orbital motion at a rate of 140 to 165 rpm.

### Embodiment 14

A shelf article comprising:
i. a shelf;
ii. a foreplate comprising a first half-collar for surrounding a pole, and a supporting member for supporting a shelf member placed thereon; and
iii. backplate comprising a second half-collar for surrounding a pole;
   wherein the fore-plate and backplate are configured to be attached to one another such that the first half-collar and the second half-collar form a channel to substantially surround the pole.

### Embodiment 15

A kit for performing a procedure of an extracorporeal blood treatment comprising the device of any one of embodiments 1-4, and a shelf article comprising:
i. a shelf;
ii. a foreplate comprising a first half-collar for surrounding a pole, and a supporting member for supporting a shelf member placed thereon; and
iii. backplate comprising a second half-collar for surrounding a pole;
   wherein the fore-plate and backplate are configured to be attached to one another such that the first half-collar and the second half-collar form a channel to substantially surround the pole.

### Embodiment 16

A kit comprising a cradle for holding a blood container securely during an orbital rotary motion about a vertical axis; and a motor for generating a circular orbital rotary motion of the cradle about a vertical axis.

### Embodiment 17

The kit of embodiment 16, that further comprises one or more adaptors attaching a cradle to the motor.

### Example—Clinical Study

A Hemodynamic Swirling Apparatus was assembled using a cradle as illustrated in FIGS. 1A, 1B and attached to an orbital vortex motor. This HSA was used to move the blood container in ozone treatments of blood of some 3000 subjects using apparatus, including the blood container, available from Hermann Apparatebau GmbH. In some instances the blood container from Zotzmann & Stahl GmbH was used.

The standard procedure instructed by Hermann Apparetebau GmbH was used, except that a HSA as described herein was used to keep the blood container in motion during the procedure. In some instances the heparin protocol below was used, which uses significantly reduced the amount of heparin than indicated by said manufacturers:
a. Allow 1000 units to be introduced into the container as the blood enters on the first pass.
b. Allow 500 units to be introduced into the container as the blood enters the container on each successive pass.
c. In the case where a filter is found at the bottom of the container, avoid emptying the container to the point where the filter is exposed to the air. Activate the filling procedure just before blood level drops to top of filter.
d. On the 7^{th} pass, if blood flow has not slowed down, and no other indication of clogging is evident, consider discontinuing the use of Heparin for the remaining 3-4 passes.

In all cases, use of heparin was substantially reduced from the amounts instructed by the manufacturers, and in some instances, the heparin protocol was omitted entirely. In all cases, incidences of clotting or clogging of the container apparatus were virtually eliminated.

Study results:
Using the Hemodynamic Swirling Apparatus, all of the High Dose Ozone IV Treatments (HDOT) attempted with 10-passes were completed using only 1 container per treatment. That is, no clogging of the blood container, outlet filter of the blood container, or tubing was observed.
Hemolysis was rarely observed. When hemolysis occurred, it was transient and trace amounts.
In 10-pass HDOT tests using the device without ozone and without heparin, no incidents of hemolysis were observed (N=70).
In tests of 10-pass HDOT using the device with ozone and without heparin, one case of trace transient hemolysis was observed (N=70)

In tests of 10-pass HDOT using the device with ozone and without heparin, 83% of the time 10-passes were completed with only 1 container, i.e., no clotting or clogging.

In view of the results above, it is reasonable that 10-pass HDOT can be performed using the Hemodynamic Swirling Apparatus with substantially less heparin. It is possible to eliminate the use of heparin altogether, perhaps then with instructions to the patient that they take aspirin the night before the procedure.

Using the HSA disclosed herein provides several advantages to an extracorporeal blood treatment protocol. Among them are:
1. savings of time in that no time is expended changing out a clogged blood container, or tubing;
2. savings of money in that only a single blood container is needed per procedure;
3. saving of money in that an IV nurse can manage 2-3 patients at once, rather than a single patient at a time; it is often found in clinical practice that users of blood containers from both Hermann Apparatebau GmbH and Zotzmann & Stahl GmbH must constantly swirl the containers manually;
4. elimination of risk of repetitive motion injury in IV nurses from constant repetitive motion of manually swirling the container;
5. heparin (anticoagulant) protocol is simplified or eliminated, at least reducing costs for heparin and providing an extracorporeal circulation that need not be opened to administer heparin;
6. reduction or elimination of risk factors to the patient due to use of heparin.

Any of the examples or embodiments described herein may include various other features in addition to or in lieu of those described above. The teachings, expressions, embodiments, examples, etc., described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

We claim:

1. A device for maintaining blood substantially free of biochemical coagulation and/or particulate precipitation, during extracorporeal treatment of the blood comprising:
i. a cradle for holding a blood container;
ii. a motor connected to the cradle for generating a circular orbital rotary motion of the cradle about a vertical axis.

2. The device of claim 1, further comprising an adaptor attaching the cradle to the motor.

3. The device of claim 1, wherein the motor is configured to operate with an orbital diameter of from approximately 6 mm to approximately 22 mm and a rotational rate of from approximately 140 rpm to approximately 165 rpm.

4. The device of claim 2, wherein the motor is configured to operate with an orbital diameter of from approximately 6 to approximately 22 mm and a rotational rate of from approximately 140 to approximately 165 rpm.

5. The device of claim 1, wherein the cradle comprises
an upper ring including a circumferential body and an aperture, the aperture being configured to hold a blood container, the upper ring further optionally including an opening in the circumferential part of the ring;
a bottom member spaced apart from the upper ring and including or having attached thereto at least one member that connects the bottom member to the motor or to an adaptor;
at least one member joining the upper ring and bottom member.

6. A method for maintaining blood in a state of laminar hemodynamic flow over a blood-contacting surface and substantially free of coagulating blood and/or particulate precipitation comprising:
a. introducing a volume of the blood into a blood container; and
b. moving the container in a rotary orbital motion to generate a sinusoidal wave in the moving blood thus establishing a laminar flow of the blood in the blood container and over the blood-contacting surface.

7. The method of claim 6, wherein the container is a rigid container having an oblate spheroid, circular cylindrical or circular conical shape.

8. The method of claim 6, in which the container has a circular cross section in a plane perpendicular to the vertical axis and through the widest portion of the container has a diameter from approximately 4 inches to approximately 6 inches, and the diameter of the rotary orbital motion is from approximately 6 mm to approximately 22 mm.

9. The method of claim 8, in which the rotary orbital motion has a rate of approximately 140 to approximately 165 rpm.

10. The method of claim 7, in which the container is one wherein a blood-contacting surface of the container comprises a glass, a polycarbonate plastic, a polyvinyl chloride plastic or a silicone-based plastic.

11. The method of claim 7, in which the blood is contacted with ozone gas.

12. In a method for extracorporeal treatment of blood, the improvement comprising maintaining the blood in a state of unidirectional hemodynamic laminar flow substantially free of biochemical coagulation activity and/or particulate precipitation by applying an orbital rotary motion to a volume of the blood to generate a sinusoidal wave in the moving blood thus establishing a laminar flow of the blood throughout the container and over a blood-contacting surface.

13. The method of claim 12, in which the orbital diameter is from approximately 6 to approximately 22 mm.

14. The method of claim 12, in which the orbital rotary motion is a circular orbital motion at a rate of approximately 140 to approximately 165 rpm.

15. The method of claim 13, in which the orbital rotary motion is a circular orbital motion at a rate of approximately 140 to approximately 165 rpm.

16. A shelf article comprising:
i. a shelf;
ii. a fore-plate comprising a first half-collar for surrounding a pole, and a supporting member for supporting a shelf member placed thereon; and
iii. a backplate comprising a second half-collar for surrounding a pole;
wherein the fore-plate and backplate are configured to be attached to one another such that the first half-collar and the second half-collar form a channel to substantially surround the pole.

17. A kit for performing a procedure of an extracorporeal blood treatment comprising the device of claim 1, and a shelf article comprising:
i. a shelf;
ii. a fore-plate comprising a first half-collar for surrounding a pole, and a supporting member for supporting a shelf member placed thereon; and
iii. backplate comprising a second half-collar for surrounding a pole;
wherein the fore-plate and backplate are configured to be attached to one another such that the first half-collar and the second half-collar form a channel to substantially surround the pole.

18. A kit for performing a procedure of an extracorporeal blood treatment comprising a cradle for holding a blood container securely during an orbital rotary motion about a vertical axis; and a motor for generating a circular orbital rotary motion of the cradle about a vertical axis.

19. The kit of claim 18 that further comprises one or more adaptors attaching a cradle to the motor.
